(19) United States
(12) Patent Application Publication
(10) Pub. No.: US 2023/0151357 A1
(43) Pub. Date: May 18, 2023

(54) METHODS AND COMPOSITIONS FOR GENOTYPING AND PHENOTYPING CELLS

(71) Applicant: Bio-Rad Laboratories, Inc., Hercules, CA (US)

(72) Inventor: Ronald LEBOFSKY, Kensington, CA (US)

(21) Appl. No.: 17/986,637

(22) Filed: Nov. 14, 2022

Related U.S. Application Data
(60) Provisional application No. 63/279,920, filed on Nov. 16, 2021.

(51) Int. Cl. C12N 15/10; C12Q 1/6841

(52) U.S. Cl. CPC C12N 15/1065; C12Q 1/6841

(57) ABSTRACT

Methods of measuring the genotype and phenotype of a cells are provided.

### CROSS-REFERENCE TO RELATED PATENT APPLICATIONS

The present patent application claims benefit of priority to U.S. Provisional Patent Application No. 63/279,920, filed Nov. 16, 2021, which is incorporated by reference for all purposes.

### BACKGROUND OF THE INVENTION

de Rutte, et al., bioRxiv Sep. 18, 2020 (https://doi.org/10.1101/2020.03.09.984245) describes “using structured microparticles, which act as suspendable and sortable microwells. These microparticles, or “nanovials”, hold single cells in sub-nanoliter volumes of fluid, 100,000 times less volume than a single well of a 1536-well microwell plate, yet require no specialized instrumentation. Fluids are easily exchanged by centrifugation and pipetting, and each compartment can be sealed and unsealed using biocompatible oils to prevent cross-talk between samples. The surfaces can be modified to bind cells or capture biomolecules for various molecular readouts.”

### BRIEF SUMMARY OF THE INVENTION

In some embodiments, the disclosure provides a method of determining the phenotype and genotype of cells. In some embodiments, the method comprises:
providing a plurality of hollow hydrophilic beads, the beads containing a cell, wherein the beads are linked to a plurality of clonal cell-barcoding oligonucleotides having a 3′ capture sequence;
contacting the beads containing the cells to an array of spots of array oligonucleotides linked to a solid planar surface, wherein the spots have clonal copies of array oligonucleotides and different spots have unique array oligonucleotides such that the sequence of the array oligonucleotides identifies the spot and wherein the array oligonucleotides comprise a 3′ end sequence that is a reverse complement of the 3′ capture sequence of the cell-barcoding oligonucleotides, wherein the spots have a size that only accommodates a single cell such that single cells reside on single spots and wherein the sequence and location of the array oligonucleotides on the array is known;
assaying the cells on the spots to determine a phenotype and recording for the spots a signal indicative of the phenotype;
releasing the oligonucleotides from the array such that released oligonucleotides from spots diffuse into beads residing on the spots, wherein a portion of the cell-barcoding oligonucleotides anneal to the array oligonucleotides;
encapsulating the beads containing cells into aqueous droplets in a water-in-oil emulsion (or otherwise introducing the beads containing cells into partitions);
tagging in the droplets (or partitions) at least one cellular nucleic acid from the cells with the cell-barcoding oligonucleotides;
nucleotide sequencing tagged cellular nucleic acids from the cell and nucleotide sequencing cell-barcoding oligonucleotides linked to the array oligonucleotides, wherein the cell barcode on the cellular nucleic acid indicates the cell origin of the cellular nucleic acid and the array oligonucleotide indicates the location of the cell on the array; and
correlating the signal on the array to the nucleotide sequence of the tagged cellular nucleic acids.

In some embodiments, after the providing and before the contacting, inducing the phenotype in the cells and sorting the beads for beads containing cells that generate the signal, thereby forming a population of beads enriched for beads containing cells, and wherein the contacting comprises contacting the population of beads enriched for beads containing cells to the array of spots of array oligonucleotides linked to the solid planar surface. In some embodiments, the signal is a fluorescent signal and the sorting comprises sorting the cells with fluorescence-activated cell sorting (FACS).

In some embodiments, the tagging comprises annealing the 3′ capture sequence of the cell-barcoding oligonucleotides to the at least one cellular nucleic acid.

In some embodiments, the 3′ capture sequence is a poly T sequence comprising at least five contiguous deoxythymidines.

In some embodiments, the 3′ capture sequence is a gene-specific capture sequence.

In some embodiments, bridge oligonucleotides are present in the droplets and the tagging comprises annealing a first end of the bridge oligonucleotide to the cell-barcoding oligonucleotides and a second end of the bridge oligonucleotide to the cellular nucleic acid from the cell.

In some embodiments, the beads are linked to at least a first set and a second set of clonal cell-barcoding oligonucleotides having a 3′ capture sequence, wherein the first set and second set have different 3′ capture sequences and wherein the 3′ capture sequence of the first set anneals to the array oligonucleotides and (i) the 3′ capture sequence of the second set anneals to the at least one cellular nucleic acid or (ii) the 3′ capture sequence of the second set anneals to a first end of a bridge oligonucleotide and a second end of the bridge oligonucleotide anneals to the at least one cellular nucleic acid.

In some embodiments, the tagging comprises ligating the cell-barcoding oligonucleotides to the at least one cellular nucleic acid.

In some embodiments, the tagging comprises primer extension wherein the cell-barcoding oligonucleotides are extended by a polymerase using the at least one cellular nucleic acid as a template.

In some embodiments, the one or more cellular nucleic acids are RNA. In some embodiments, the tagging comprises reverse transcription.

In some embodiments, the one or more cellular nucleic acids are DNA.

In some embodiments, the cells are B cells and the at least one cellular nucleic acid from the cells encodes at least a portion of an antibody variable region.

In some embodiments, the cells are T-cells and the at least one cellular nucleic acid from the cells encodes at least a variable portion of a T-cell receptor.

In some embodiments, the assaying comprises in situ immunofluorescence, in situ immunohistochemistry, or in situ hybridization to produce the signal.

In some embodiments, the assaying comprises adding target cells to the array and assaying the ability of the cells in the hollow hydrophilic beads to alter a phenotype of the target cells.

In some embodiments, the beads containing the cells are encapsulated in a droplet before the assaying. In some embodiments, a second encapsulation occurs after the assaying. In some embodiments, the beads containing the cells are encapsulated only after the assaying.

### BRIEF DESCRIPTION OF THE DRAWINGS

FIG. 1 depicts use of core-empty gel beads (i.e., hollow hydrophilic beads), loading of cells into the gel beads, inducing a phenotype in the cells to generate a fluorescent signal and sorting the beads in a cell sorter based on fluorescence. As noted herein, inducing the phenotype or otherwise sorting the beads for those cells that contain cells and preferably that generate a fluorescent signal indicative of the desired phenotype is optional but can be beneficial focusing resources on beads that are most likely to be of interest. In some embodiments, the desired phenotype is the ability of a cell to express and optionally secrete a product (e.g., an antibody or non-antibody protein), which in some embodiments can be detected by a fluorescent reagent or dye.

FIG. 2 continues a workflow from FIG. 1 and depicts binding of gel beads (which optionally have been pre-sorted as depicted in FIG. 1) to oligonucleotide spots on a 2D array. Imaging can be used to detect the phenotype signal from the cells in beads on the array. The sequences and position of the array oligonucleotides on the array is known allowing one to correlate the signal to the array oligonucleotide sequences. Following detecting of the signal, the array oligonucleotides are released from the array and bound to complementary oligonucleotides on the gel bead.

FIG. 3 depicts exemplary sequences and molecular interactions between core-empty gel beads and array oligonucleotides in an embodiment in which the target cellular nucleic acids are polyA RNA from the cells.

FIG. 4 depicts single cell/bead encapsulation and barcoding. Core-empty beads containing cells and bound array oligonucleotides are encapsulated in oil through the addition of oil and physical agitation in a process known as “particle templated emulsification”. After encapsulation, cells are lysed, bead barcode oligonucleotides are optionally released from the core-empty beads followed by barcoding of both the cellular nucleic acids and the array oligonucleotides. Samples are then prepared for sequencing followed by sequencing. The core-empty bead barcode provides the information to group together reads from a single cell. The array oligonucleotides associated with the core-empty barcodes report on the original location in the 2D array. Thus, by virtue of sharing the same core-empty barcode, the single cell substrates can be linked to a physical location on the 2D array.

FIG. 5 depicts use of two aqueous inlet droplet maker to encapsulate gel beads. Notably, because each core-empty bead contains a single barcode and cells are bound to the bead, all droplets that contain a bead will also contain a cell. Droplets that do not have a core-empty bead will not have a cell as the cells are only bound to beads. Thus to ensure a one-to-one ratio of beads to cells, droplets are loaded at low mean number of beads/droplet, i.e. X.=0.05-0.15 to prevent the co-occurrence of 2 beads per droplets. Thus, no bead beating Poisson is required.

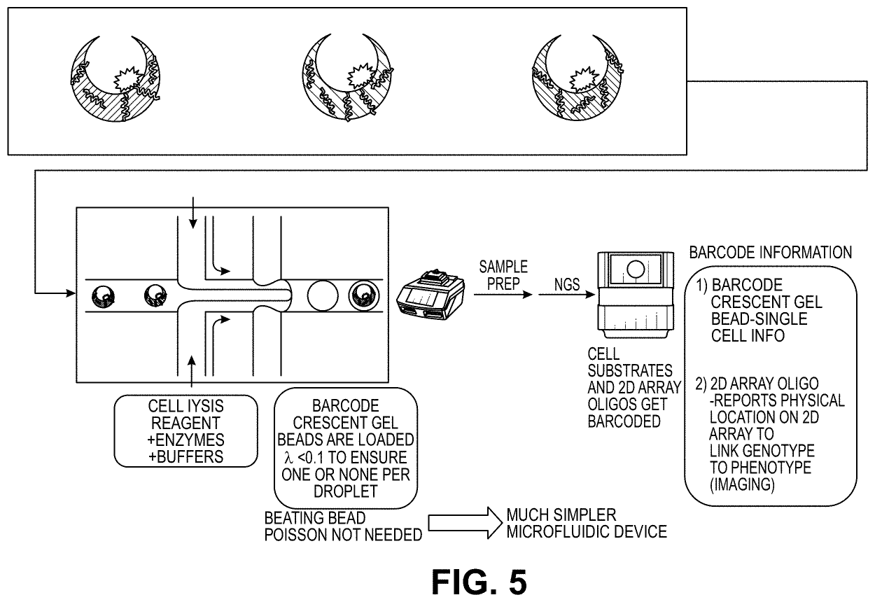

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization described below are those well-known and commonly employed in the art. Standard techniques are used for nucleic acid and peptide synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well-known and commonly employed in the art.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a bead” includes a plurality of such beads and reference to “the sequence” includes reference to one or more sequences known to those skilled in the art, and so forth.

An “oligonucleotide” is a polynucleotide. Generally oligonucleotides will have fewer than 250 nucleotides, in some embodiments, between 4-200, e.g., 10-150 nucleotides.

The term “amplification reaction” refers to any in vitro means for multiplying the copies of a target sequence of nucleic acid in a linear or exponential manner. Such methods include but are not limited to polymerase chain reaction (PCR); DNA ligase chain reaction (see U.S. Pat. Nos. 4,683,195 and 4,683,202; PCR Protocols: A Guide to Methods and Applications (Innis et al., eds, 1990)) (LCR); QBeta RNA replicase and RNA transcription-based amplification reactions (e.g., amplification that involves T7, T3, or SP6 primed RNA polymerization), such as the transcription amplification system (TAS), nucleic acid sequence based amplification (NASBA), and self-sustained sequence replication (3SR); isothermal amplification reactions (e.g., single-primer isothermal amplification (SPIA)); as well as others known to those of skill in the art.

“Amplifying” refers to a step of submitting a solution to conditions sufficient to allow for amplification of a polynucleotide if all of the components of the reaction are intact. Components of an amplification reaction include, e.g., primers, a polynucleotide template, polymerase, nucleotides, and the like. The term “amplifying” typically refers to an “exponential” increase in target nucleic acid. However, “amplifying” as used herein can also refer to linear increases in the numbers of a select target sequence of nucleic acid, such as is obtained with cycle sequencing or linear amplification. In an exemplary embodiment, amplifying refers to PCR amplification using a first and a second amplification primer.

As used herein, “nucleic acid” means DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, points of attachment and functionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications including but not limited to capping with a fluorophore (e.g., quantum dot) or another moiety.

The term “sample nucleic acid” refers to a polynucleotide such as DNA, e.g., single stranded DNA or double stranded DNA, RNA, e.g., mRNA or miRNA, or a DNA-RNA hybrid. DNA includes genomic DNA and complementary DNA (cDNA).

A nucleic acid, or a portion thereof, “hybridizes” to another nucleic acid under conditions such that non-specific hybridization is minimal at a defined temperature in a physiological buffer (e.g., pH 6-9, 25-150 mM chloride salt). In some cases, a nucleic acid, or portion thereof, hybridizes to a conserved sequence shared among a group of target nucleic acids. In some cases, a primer, or portion thereof, can hybridize to a primer binding site if there are at least about 6, 8, 10, 12, 14, 16, or 18 contiguous complementary nucleotides, including “universal” nucleotides that are complementary to more than one nucleotide partner. Alternatively, a primer, or portion thereof, can hybridize to a primer binding site if there are fewer than 1 or 2 complementarity mismatches over at least about 12, 14, 16, or 18 contiguous complementary nucleotides. In some embodiments, the defined temperature at which specific hybridization occurs is room temperature. In some embodiments, the defined temperature at which specific hybridization occurs is higher than room temperature. In some embodiments, the defined temperature at which specific hybridization occurs is at least about 37, 40, 42, 45, 50, 55, 60, 65, 70, 75, or 80° C. In some embodiments, the defined temperature at which specific hybridization occurs is 37, 40, 42, 45, 50, 55, 60, 65, 70, 75, or 80° C. For hybridization to occur, the primer binding site and the portion of the primer that hybridizes will be at least substantially complementary. By “substantially complementary” is meant that the primer binding site has a base sequence containing an at least 6, 8, 10, 15, or 20 (e.g., 4-30, 6-30, 4-50) contiguous base region that is at least 50%, 60%, 70%, 80%, 90%, or 95% complementary to an equal length of a contiguous base region present in a primer sequence. “Complementary” means that a contiguous plurality of nucleotides of two nucleic acid strands are available to have standard Watson-Crick base pairing. For a particular reference sequence, 100% complementary means that each nucleotide of one strand is complementary (standard base pairing) with a nucleotide on a contiguous sequence in a second strand.

As used herein a “barcode” is a short nucleotide sequence (e.g., at least about 4, 6, 8, 10, 12, 15, 20, 50 or 75 or 100 nucleotides long or more) that identifies a molecule to which it is conjugated or from the partition in which it originated. Barcodes can be used, e.g., to identify molecules originating in a partition, bead, or spot as later sequenced from a bulk reaction. Such a barcode can be unique for that partition, bead or spot as compared to barcodes present in other partitions, bead or spot. For example, partitions containing target RNA from single-cells can be subject to reverse transcription conditions using primers that contain different partition-specific barcode sequence in each partition, thus incorporating a copy of a unique “cellular barcode” (because different cells are in different partitions and each partition has unique partition-specific barcodes) into the reverse transcribed nucleic acids of each partition. Thus, nucleic acid from each cell can be distinguished from nucleic acid of other cells due to the unique “cellular barcode.”

In some embodiments described herein, barcodes described herein uniquely identify the molecule to which it is conjugated, i.e., the barcode acts as a unique molecular identifier (UMI).

The length of the underlying barcode sequence determines how many unique samples can be differentiated. For example, a 1 nucleotide barcode can differentiate 4, or fewer depending on degeneracy, different partitions; a 4 nucleotide barcode can differentiate 4⁴ or 256 partitions or less; a 6 nucleotide barcode can differentiate 4096 different partitions or less; and an 8 nucleotide barcode can index 65,536 different partitions or less.

As used herein, the term “partitioning” or “partitioned” refers to separating a sample into a plurality of portions, or “partitions.” Partitions are generally physical, such that a sample in one partition does not, or does not substantially, mix with a sample in an adjacent partition. Partitions can be solid or fluid. In some embodiments, a partition is a solid partition, e.g., a microchannel or well. In some embodiments, a partition is a fluid partition, e.g., a droplet. In some embodiments, a fluid partition (e.g., a droplet) is a mixture of immiscible fluids (e.g., water and oil). In some embodiments, a fluid partition (e.g., a droplet) is an aqueous droplet that is surrounded by an immiscible carrier fluid (e.g., oil).

“Phenotype” refers to any physical aspect of a cell that can be detected. For example, the phenotype can be expression of a particular protein or ability of the cell to trigger a biological response. A phenotype can be measured in some embodiments using one or more reporters that produce a signal that can be measured and that is proportional to the phenotype.

“Genotype” refers to one or more genomic sequences in a cell. In some embodiments, the genotype is linked to the phenotype. For example, the phenotype may be the ability of a B-cell to produce an antibody of a desired specificity and the genotype can be the nucleotide sequence encoding the variable domain(s) of the antibody produced by the B-cells.

“Clonal” copies of a polynucleotide means the copies are identical in sequence. In some embodiments, there are at least 100, 1000, 10⁴ or more clonal copies of oligonucleotides in a spot on an array or linked to a hydrophilic bead.

Oligonucleotides can be linked to a solid support by covalent linkages or non-covalent linkages, e.g., electrostatic linkages when two nucleic acids anneal.

An “array” is an ordered plurality of items. The term can refer to an ordered plurality of oligonucleotides, or the ordered array linked to a solid surface that is optionally planar. “ordered” refers to known locations on the array and is not intended to indicate a particular alignment of the items, though in some embodiments the items can be in a grid.

A 3′ capture sequence on an oligonucleotide refers to the 3′ most portion of an oligonucleotide. The capture sequence can be as few as 1-2 nucleotides in length but is more commonly 6-12 nucleotides in length and in some embodiments is 4-20 or more nucleotides in length. The capture sequence can be completely complementary to a target nucleic acid (e.g., the 3′ end of the target nucleic acid), though as will be appreciated in some embodiments and certain conditions, 1, 2, 3, 4, or more nucleotides may be mismatched while still allowing the 3′ capture sequence of an oligonucleotide anneal to the target nucleic acid. In other embodiments, conditions can be selected such that only completely complementary sequences will anneal.

“Tagging” a nucleic acid refers to linking the nucleic acid with another tagging polynucleotide, for example a tagging polynucleotide comprising one or more barcode sequences. Tagging can be covalent (e.g., via ligation or by primer extension) or non-covalent (via Watson-Crick base pairing only).

A PCR handle sequence” refers to a sequence that can be added to target nucleic acids such that an entire set of different target nucleic acids can be amplified later with a universal primer.

### DETAILED DESCRIPTION OF THE INVENTION

Methods of genotyping and phenotyping a plurality of cells are provided. The method described herein can comprise use of hollow hydrophilic beads containing cells (e.g., one cell per bead) and allows for assaying the cells in the beads for a phenotype and determining genotype information from the same cells (e.g., in some embodiments without expansion of the cells). The methods involve placing each bead on an array of oligonucleotide “spots” such that each spot is associated with one bead. The cells in the beads on the array can be assayed for a desired phenotype (which can be optionally detected optically on the array) and then the oligonucleotides from the array are released from the array. Due to the proximity of the bead on the spot (compared to other cells), the bead in proximity to the spot will receive all or at least a majority of the released oligonucleotides (at least relative to other beads), which in turn anneal to complementary polynucleotides associated with the bead. This will provide a link that can be assayed later by sequencing to associate the spot (and detected phenotype from that spot) with sequencing information from the cell that resided on the spot. Primers associated with the bead, which can be the complementary polynucleotides noted above or separate sequences, and having one or more identifying barcode can be used to prime primer extension reactions with nucleic acids from the cell as templates to generate cellular nucleic acids having PCR handle sequences and barcode information for nucleotide sequencing. The resulting sequencing information will generate: target nucleic acids with bead barcodes as well as bead barcodes associated with array oligonucleotides, thereby allowing one to associate phenotype information from the array with genotype information for the cells.

Hollow hydrophilic beads can be used to capture cells within. Exemplary hollow hydrophilic beads are described in, for example, de Rutte, et al., bioRxiv Sep. 18, 2020 (https://doi.org/10.1101/2020.03.09.984245) and U.S. Patent Publication No. US 2021/0268465 (describing the hollow beads as “dropicles” or “drop-carrier particles”). The hollow beads can have a diameter, for example, of between about 10 μm and about 500 μm, e.g., between 20 μm and 200 μm, e.g., between 40 and 20 μm. In some embodiments, the hollow beads are crescent shape such that they have a tendency to settle under gravity with their openings up. The beads include a void volume or cavity therein. The void or cavity delimits the three-dimensional volume that holds at least a portion of a dispersed phase solution or fluid (e.g., aqueous phase). Exemplary fluid volumes held within the void or cavity can be for example about 100 fL-10 nL. A length dimension (e.g., diameter for spherical void) of the void or cavity within a bead can be several microns, e.g., more than about 5 μm and less than about 250 μm. Generally one phase of the ATPS comprises a crosslinkable component, while the other phase does not contain the crosslinkable component. In some embodiments a crosslinkable PEG phase and dextran phase are co-flowed in a microfluidic droplet generator device along with a third oil phase containing surfactant to generate mixed aqueous emulsions in which a uniform fraction of PEG phase and dextran phase is present in each droplet suspended in an oil phase. Microgel beads containing cavities may be fabricated utilizing aqueous two-phase systems (ATPS) combined with droplet microfluidics. See, e.g., U.S. Patent Publication No. US 2021/0268465; S. Ma, et al. Small. 8, 2356-2360 (2012); B. D. Fairbanks, et al., A versatile synthetic extracellular matrix mimic via thiol-norbornene photopolymerization. Adv. Mater. (2009).

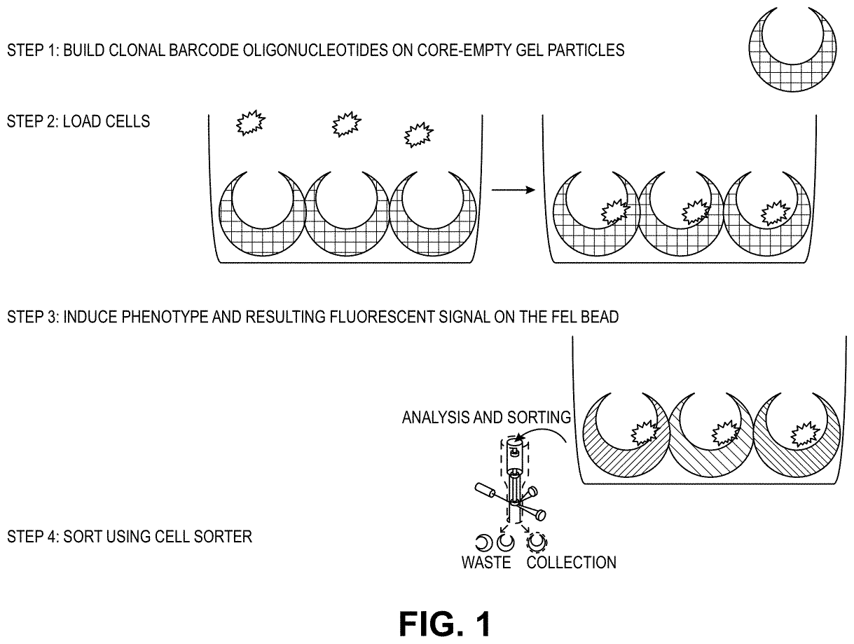

Cells can be introduced into the beads as desired. This aspect is depicted in FIG. 1. In some embodiments, cells can be introduced into the beads by preparing a layer of beads with openings pointing upward (which can occur due to weighting of crescent-shaped beads) and a cell suspension and allowing cells from the suspension to enter the openings of the beads by gravity.

Any type of cells can be used. In some embodiments, the cells are eukaryotic cells, e.g., human, mouse, rat or other mammalian cells. In some embodiments, the cells are immune cells, e.g., including but not limit to hybridomas, T-cells or B-cells.

In the methods described herein the hollow hydrophilic beads are linked or otherwise associated with a plurality of clonal cell-barcoding oligonucleotides having a 3′ capture sequence. The cell-barcoding oligonucleotides can be linked as desired to the beads. Methods of linking the cell-barcoding oligonucleotides to the beads will depend on the composition of the beads. In some embodiments, the 5′ end of the cell-barcoding oligonucleotides is linked to the beads or the cell-barcoding oligonucleotides are otherwise linked such that the 3′ capture sequence is available for hybridization to complementary sequences.

In some embodiments, the beads can be linked to two or more sets of cell-barcoding oligonucleotides, wherein each set includes multiple clonal copies (e.g., 10⁴, 10⁵, 10⁶ or more). For example, in some embodiments, one set includes a 3′ capture sequence complementary to the array oligonucleotides as detailed further below and one or more different sets include capture sequences specific for a particular target sequence, e.g., a gene or mRNA sequence. In embodiments in which multiple sets of cell-barcoding oligonucleotides are linked to the same beads, all sets will include the same cell (bead-specific) barcode such that all nucleic acids linked during the method to the cell-barcoding oligonucleotides from one bead are tagged with the same cell-specific barcode, indicating origination from the contents of the bead.

Optionally, in some embodiments, one can enrich the population of beads for those that contain cells, or those that contain cells that have at least a threshold level of detectable phenotype. See, e.g., FIG. 1. This can be advantageous as it avoids an excess of beads that are empty or contain cells that are not of interest, allowing reagents to be used on beads and/or cells that are more likely to be of interest. As discussed in more detail below, in some embodiments, the desired phenotype can be fluorescence or other optically-detectable signal. This can result for example in embodiments in which a fluorescent reporter gene is linked to a promoter indicative of the desired phenotype or in embodiments in which fluorescently-labeled binding molecules indicate the presence of a desired product by the cell. In embodiments in which fluorescence is an output of the cells, fluorescence-activate cell sorting (FACS) can be applied to the cells to enrich the population of beads containing cells for those with at least a threshold amount of fluorescence. Alternatively, other sorting method scan be used depending on how the phenotype is manifested to enrich the population for beads containing cells of potential interest.

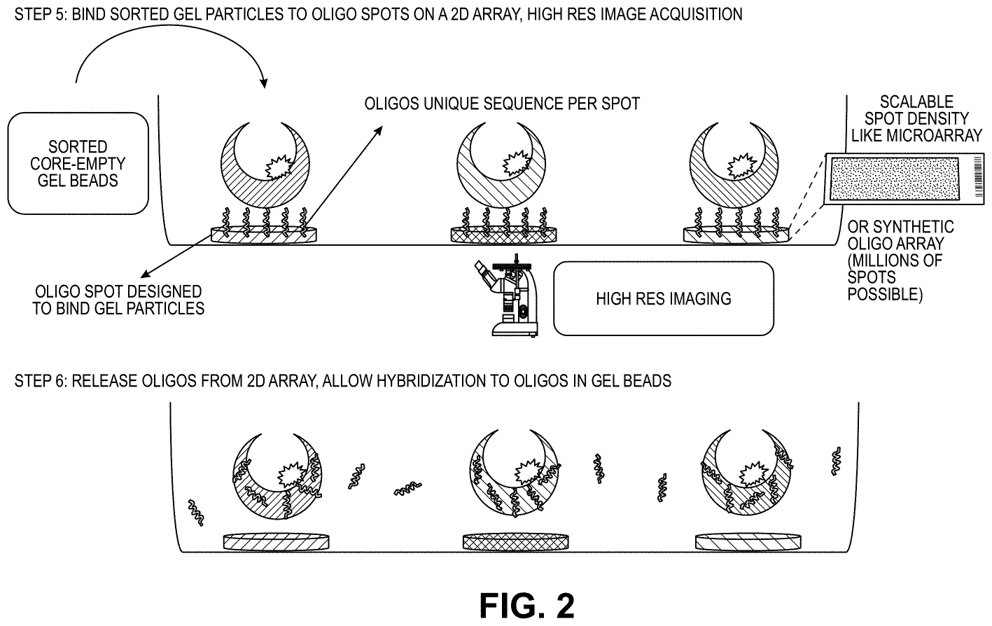

Whether the population of beads containing cells has been sorted as described above or not, the population can be applied to a planar array of oligonucleotides (“array oligonucleotides”). See, e.g., FIG. 2. The array can be designed and generated such that clonal copies of oligonucleotides are provided in spots on the array. Each spot will have different oligonucleotides (at least differing by a barcode sequence in the oligonucleotide sequences) such that each spot's oligonucleotides are unique for that spot, allowing one to use the sequence of the clonal oligonucleotides at the spot to identify the position of the bead at that spot. As used herein, the term “spot” refers to a defined area of the array at which a particular set of clonal oligonucleotides reside. The spot size will be sufficiently small in size compared to the size of the beads such that only one bead can reside on any particular spot, allowing for subsequent diffusion of clonal array oligonucleotides from the spot to a single cell as described herein and not substantially contaminating adjacent beads.

Methods of generating arrays of oligonucleotides with known sequences are known and can be used to generate the arrays described herein. See, for example, US2021/0332351 and US2020/0299322 or as otherwise described by for example DNA Script or Twist Biosciences. These methods, can for example, provide for spatially addressable oligonucleotides on a planar array, meaning that the oligonucleotide sequences at each spot on the array are known. In some embodiments, such planar supports have a plurality of sites comprising at least 256 sites, at least 512 sites, at least 1024 sites, at least 5000 sites, at least 10,000 sites, at least 25,000 sites, or at least 100,000 sites and as many as 10,000,000 sites. In some embodiments, the discrete site at which synthesis of spots take place each has an area in the range of from 0.25 μm² to 1000 μm², or from 1 μm² to 1000 μm², or from 10 μm² to 1000 μm², or from 100 μm² to 1000 μm². In some embodiments, the amount of polynucleotides synthesized at each spot is at least 10⁻⁶ fmol, or at least 10⁻³ fmol, or at least 1 fmol, or at least 1 pmol, or the amount of polynucleotide synthesized at each spot is in the range of from 10⁻⁶ fmol to 1 fmol, or from 10⁻³ fmol to 1 fmol, or from 1 fmol to 1 pmol, or from 10⁻⁶ pmol to 10 pmol, or from 10⁻⁶ pmol to 1 pmol. In some embodiments, the number of polynucleotides synthesized at each spot is in the range of from 1000 molecules to 10⁶ molecules, or from 1000 molecules to 10⁹ molecules, or from 1000 molecules to 10¹² molecules. In some embodiments, the array is on a flow cell.

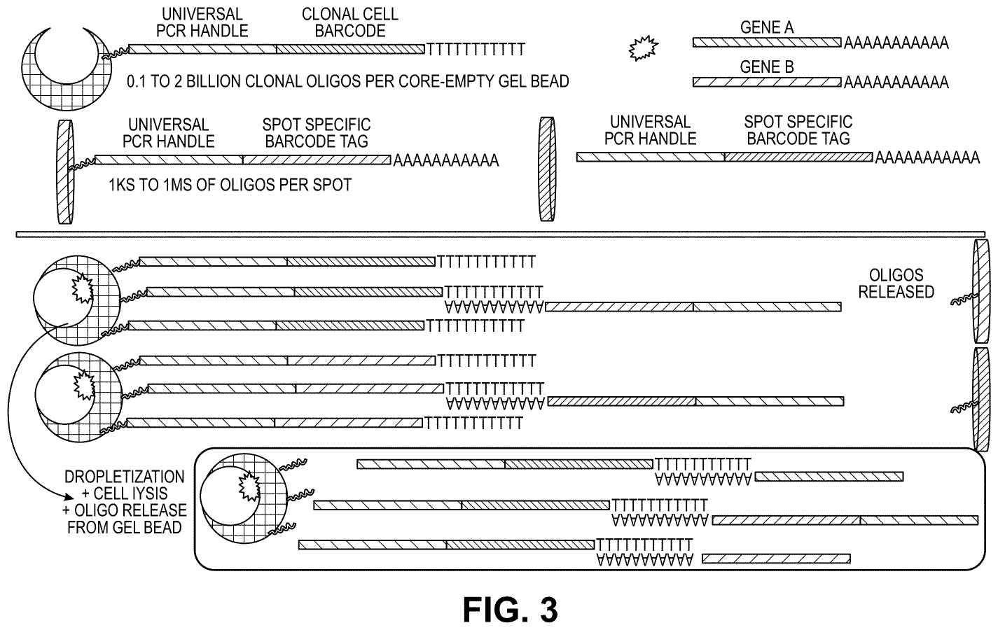

The array oligonucleotides will comprise at least a 3′ sequence and an array spot barcode sequence, wherein the array spot barcode sequence is unique for the particular spot. See, e.g., FIG. 3. The 3′ sequence will be of sufficient length and sequence to anneal to, in a later step, one or more of the cell-barcoding oligonucleotide 3′ end capture sequences, or alternatively to a splint oligonucleotide that can anneal to both the cell-barcoding oligonucleotide 3′ end capture sequence and the array oligonucleotide 3′ sequence. Thus in some embodiments, the array oligonucleotide 3′ sequence is the reverse complement of the cell-barcoding oligonucleotide 3′ end capture sequence. In some embodiments, the length of the array oligonucleotide 3′ sequence is 4-20 nucleotides, e.g., 8-15 nucleotides. The length of the array spot barcode sequence will be sufficient to provide unique barcodes between spots and thus can depend on the number of unique spots. In some embodiments, the barcodes differ by at least two nucleotides from the closest (as measured by aligned sequence identity not physical proximity) barcode sequence in the array.

Beads containing cells can be associated to spots by providing a suspension of the beads to the array and allowing the beads to settle on spots on the array. In one embodiment, the array is designed to have physical chambers, such as microwells, with oligonucleotide-containing spots located at the base of the chamber. The chambers' size allows for the capture of a single bead only. In another embodiment, the spots contain molecules that bind the hydrogel bead. For example if the hydrogel bead contains cross-linked biotin, and the spots contain streptavidin, and the beads will bind to the spots on the array through biotin-streptavidin linkage.

Once the beads reside on the spots on the array, a phenotype of the cells can be measured. See, e.g., FIG. 2. The position on the array (or identity of the oligonucleotide at that position, or both) and the signal from the phenotype can be recorded, e.g., in memory of a computer. Because the oligonucleotide sequence and location of the spots is known, the sequence of the oligonucleotide on the spot can be associated with the signal detected at the spot.

The phenotype can be detected as desired. In some embodiments, immunohistochemical detection is used to detect the presence, absence or amount of one or more cell surface or secreted proteins associated with a cell in the bead, or one or more protein or RNA expressed in the cell. In some embodiments for example, the cells are lysed in the beads and then the phenotype detected is the presence or amount of one or more target protein or nucleic acid. In some embodiments, the cells in the beads are stained, optionally for example with fluorescent or bright field staining. In some embodiments, the cells on the array can be contacted with one or more probe that specifically or non-specifically detects a target nucleic acid or protein form the cell. Exemplary probes can include but are not limited to antibodies, non-antibody proteins (e.g., ligands or antigen targets in embodiments in which the cells produce an antibody), nucleic acids, aptamers, RNA-guided nucleases (which may include one or more mutation reducing or eliminating their nuclease activity) such as but not limited to Cas9 and dCas9. In some embodiments, the cells are hybridomas, B-cells or T-cells and the phenotype detected is the ability of antibodies from the hybridomas and B-cells or the T-cell receptor (TCR) on the T-cells to bind to a target antigen. Any probe can be directly or indirectly labeled such that specific binding can be detected. One or more appropriate washes and/or application of blocking agents, can be performed to reduce background before detection of signal. Signal representing the phenotype can be detected for example by a microscope, e.g., a fluorescence microscope or epifluorescence microscope. One example of phenotypic information that can be detected in this fashion is real time acquisition of antibody secretion and binding kinetics. This may involve for example a homogenous immunoassay that does not require consecutive washing and staining steps. Other phenotypic assays are described in, for example, Gonzales-Munoz, et al., Drug Discovery Today, 2016. These assays may involve cell-to-cell or cell-to-organoid functions including agonist or antagonist effects on cell signaling, proliferation, and/or apoptosis. The assays may also involve the neutralization of viral or bacterial infection.

In some embodiments, the phenotype assayed is related to cell function or survival. The cell function or survival can be that of the cell assayed, or can be a second “target” cell added as part of the assay for phenotype. For example, the cell in the bead (and whose nucleic acids will be sequenced) can in some embodiments alter a second cell present in the assay such that the phenotype of the second cell is assayed. For example, in some embodiments, the phenotype is cell killing, cell immortality (the opposite of killing), cell stemmness, cell differentiation, cell adhesion, cell aggregation, cell secretion (secondary secretion product, e.g., not necessarily the one being released from the initial cell of interest) and/or cell senescence. In one embodiment, T-cells are in the hydrophilic hollow beads and target cells are added in the assay to monitor the ability of the T-cells to lyse the target cells. In some embodiments, the beads containing cells are encapsulated in droplets prior to contacting the beads to spots on the array. This can be especially useful in aspects in which the array allows for wettability, allowing the droplet to be disrupted at the interface with the surface of the array. The oligonucleotides can be subsequently released into the droplet and allowed to bind to the bead.

The array oligonucleotides, with beads still residing on the spots, can be released from the array before, during or after performing an assay on the array to detect a desired phenotype, resulting in the array oligonucleotides diffusing into the adjacent bead. See, e.g., FIG. 2. Releasing the array oligonucleotides before or during performance of the phenotype assay requires a mechanism to hold the bead in place on arrays spots that does not depend on the array oligonucleotides themselves. In some embodiments, in view of the reverse complementary 3′ sequences of the array oligonucleotides and the cell-barcoding oligonucleotides, these oligonucleotides can anneal, significantly preventing or inhibiting diffusion of the cleaved array oligonucleotides from diffusing into adjacent beads. In some embodiments, the array oligonucleotides are released from the array by cleaving the oligonucleotides with an enzyme, e.g., a restriction enzyme. In some embodiments, the oligonucleotides can be linked to the array by a cleavable linker and the array can be exposed to conditions that cleave the cleavable linker. For example, the oligonucleotides may be linked to the array through a di-sulfide bond, whereby the bond may be cleaved through application of a reducing agent such as DTT or TCEP. Another example is that array oligonucleotides may contain dUTPs in the bases closest to the solid support, whereby the application of USER enzyme will release the oligonucleotide.

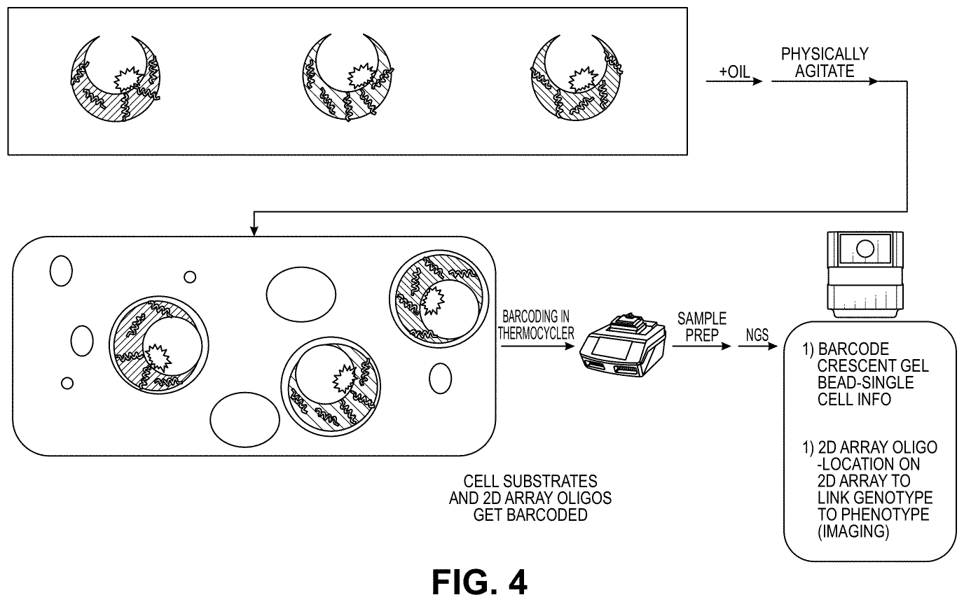

Following annealing of the released array oligonucleotides onto oligonucleotides associated (e.g., linked) to the beads, the beads can be removed from the array and inserted into partitions (e.g., droplets or wells). See, e.g., FIG. 4. In some embodiments, the beads are encapsulated into aqueous droplets in a water-in-oil emulsion. Methods and compositions for partitioning a sample are described, for example, in published patent applications WO 2010/036,352, US 2010/0173,394, US 2011/0092,373, and US 2011/0092,376, the contents of each of which are incorporated herein by reference in the entirety. The plurality of mixture partitions can be in a plurality of emulsion droplets, or a plurality of wells, etc. The partitions can be picowells, nanowells, or microwells. The mixture partitions can be pico-, nano-, or micro-reaction chambers, such as pico, nano, or microcapsules. The mixture partitions can be pico-, nano-, or micro-channels. The mixture partitions can be droplets, e.g., emulsion droplets.

In some embodiments, the beads are added to well partitions in an array of wells. For example in some embodiments, the wells of an array are sized such that the wells allow for one but only one bead in a well. However, in some embodiments, the size of the wells can be large enough to accept up to 2, 3, 4, 5, 6, 7, 8, 9, or 10 solid supports per well. The wells of the array can be a combination of wells of different depths such that no more than a quantum (0, 1, 2, 3, 4, 5, etc.) number of beads of the same size can be accommodated in the microwells and the location for each type of microwell of a certain depth is pre-determined. Exemplary array of wells and well descriptions can be found for example in U.S. Pat. Nos. 9,103,754 and 10,391,493. The array of wells (set of nanowells, microwells, wells) functions to capture the solid supports, optionally in addressable, known locations. As such, the array of wells is preferably configured to facilitate bead capture in at least one of a single-solid support format or optionally in small groups of solid supports. Exemplary microwell arrays and methods of delivery of beads to the microwells and analysis thereof is described in, e.g., PCT/US2021/034152.

Once the beads are in partitions, nucleic acids from the cells within the beads can be tagged by linking the cell-barcoding oligonucleotides to one or more target nucleic acid from the cell. See, e.g., FIG. 3, bottom portion. The target nucleic acids can be for example, RNA, DNA (e.g., genomic DNA) or both. In embodiments in which the target nucleic acid is RNA (e.g., mRNA) the cell-barcoding oligonucleotides can be used a RT primers to form a first strand cDNA via reverse transcription. In some embodiments, a second strand cDNA is also formed. Alternatively, in embodiments in which the target nucleic acids are DNA, the cell-barcoding oligonucleotides can prime primer extension products comprising at least a portion of the target nucleic acid sequence. In other embodiments, the cell-barcoding oligonucleotides are ligated to target nucleic acids. In some embodiments the cells are lysed prior to barcoding. In some embodiments, cells are fixed and barcoding occurs in situ. In some embodiments, the bead oligonucleotides are released from the beads prior to barcoding.

As noted above, in some embodiments, the 3′ end sequences of the cell-barcoding oligonucleotides anneal directly to the target nucleic acids. For example, if the target nucleic acids are mRNA, the 3′ end sequence can be a poly dT sequence (e.g., 6-20 contiguous dT nucleotides), or the 3′ end can include randomer (e.g., random sequences of 6-10 or more nucleotides) to randomly prime targets, or the 3′ end sequences can be gene-specific to specifically amplify one or more target nucleic acids. Similarly for DNA nucleic acid target sequences, the 3′ end sequence can include randomer (e.g., random sequences of 6-10 or more nucleotides) to randomly prime targets, or the 3′ end sequences can be gene-specific to specifically amplify one or more target nucleic acids. In other embodiments, one or more splint oligonucleotides (which can be double-stranded or single-stranded) can be added to, or included in, the partitions and the splint oligonucleotides can anneal to the 3′ end of the cell-barcoding oligonucleotides and anneal to a target nucleic acid. In some embodiments, two or three or more different splint oligonucleotides can be used with each targeting a different target nucleic acid sequence.

Once the nucleic acids have been tagged with the cell-barcoding oligonucleotides, the tagged nucleic acids can be prepared for nucleotide sequencing as desired. For example, universal priming sequences can be added on both ends of the tagged sequences (one universal priming sequence, also referred to as a “PCR handle” being supplied by the cell-barcoding oligonucleotides.

In some embodiments, one or more reagents (e.g., one or more amplification primer(s), probe(s), enzyme(s), oligonucleotides or a combination thereof) can be introduced into the plurality of partitions. Methods and compositions for delivering reagents to one or more partitions include microfluidic methods as known in the art; droplet or microcapsule merging, coalescing, fusing, bursting, or degrading (e.g., as described in U.S. 2015/0027,892; US 2014/0227,684; WO 2012/149,042; and WO 2014/028,537); droplet injection methods (e.g., as described in WO 2010/151,776); and combinations thereof.

In some embodiments, a droplet is an aqueous droplet that is surrounded by an immiscible carrier fluid (e.g., oil). In some embodiments, a droplet is an oil droplet that is surrounded by an immiscible carrier fluid (e.g., an aqueous solution). In some embodiments, the droplets are relatively stable and have minimal coalescence between two or more droplets. In some embodiments, less than 0.0001%, 0.0005%, 0.001%, 0.005%, 0.01%, 0.05%, 0.1%, 0.5%, 1%, 2%, 3%, 4%, 5%, 6%, 7%, 8%, 9%, or 10% of droplets generated from a sample coalesce with other droplets. The emulsions can also have limited flocculation, a process by which the dispersed phase comes out of suspension in flakes. Methods of emulsion formation are described, for example, in published patent applications WO 2011/109546 and WO 2012/061444, the entire content of each of which is incorporated by reference herein.

In some embodiments, the droplet is formed by flowing an oil phase through an aqueous sample comprising the sample and reaction components. The oil phase may comprise a fluorinated base oil which may additionally be stabilized by combination with a fluorinated surfactant such as a perfluorinated polyether. In some embodiments, the base oil comprises one or more of a HFE 7500, FC-40, FC-43, FC-70, or another common fluorinated oil. In some embodiments, the oil phase comprises an anionic fluorosurfactant. In some embodiments, the anionic fluorosurfactant is Ammonium Krytox (Krytox-AS), the ammonium salt of Krytox FSH, or a morpholino derivative of Krytox FSH. Krytox-AS may be present at a concentration of about 0.1%, 0.2%, 0.3%, 0.4%, 0.5%, 0.6%, 0.7%, 0.8%, 0.9%, 1.0%, 2.0%, 3.0%, or 4.0% (w/w). In some embodiments, the concentration of Krytox-AS is about 1.8%. In some embodiments, the concentration of Krytox-AS is about 1.62%. Morpholino derivative of Krytox FSH may be present at a concentration of about 0.1%, 0.2%, 0.3%, 0.4%, 0.5%, 0.6%, 0.7%, 0.8%, 0.9%, 1.0%, 2.0%, 3.0%, or 4.0% (w/w). In some embodiments, the concentration of morpholino derivative of Krytox FSH is about 1.8%. In some embodiments, the concentration of morpholino derivative of Krytox FSH is about 1.62%.

In some embodiments, the oil phase further comprises an additive for tuning the oil properties, such as vapor pressure, viscosity, or surface tension. Non-limiting examples include perfluorooctanol and 1H,1H,2H,2H-Perfluorodecanol. In some embodiments, 1H,1H,2H,2H-Perfluorodecanol is added to a concentration of about 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.25%, 1.50%, 1.75%, 2.0%, 2.25%, 2.5%, 2.75%, or 3.0% (w/w). In some embodiments, 1H,1H,2H,2H-Perfluorodecanol is added to a concentration of about 0.18% (w/w).

In some embodiments, the emulsion is formulated to produce highly monodisperse droplets having a liquid-like interfacial film that can be converted by heating into microcapsules having a solid-like interfacial film; such microcapsules may behave as bioreactors able to retain their contents through an incubation period. See, e.g., U.S. Pat. No. 10,378,048. The conversion to microcapsule form may occur upon heating. For example, such conversion may occur at a temperature of greater than about 40°, 50°, 60°, 70°, 80°, 90°, or 95° C. During the heating process, a fluid or mineral oil overlay may be used to prevent evaporation. Excess continuous phase oil may or may not be removed prior to heating. The biocompatible capsules may be resistant to coalescence and/or flocculation across a wide range of thermal and mechanical processing. Following conversion, the microcapsules may be stored at about −70°, −20°, 0°, 3°, 4°, 5°, 6°, 7°, 8°, 9°, 10°, 15°, 20°, 25°, 30°, 35°, or 40° C.

The microcapsule partitions, which may contain one or more polynucleotide sequences and/or one or more one or more sets of primers, may resist coalescence, particularly at high temperatures. Accordingly, the capsules can be incubated at a very high density (e.g., number of partitions per unit volume). In some embodiments, greater than 100,000, 500,000, 1,000,000, 1,500,000, 2,000,000, 2,500,000, 5,000,000, or 10,000,000 partitions may be incubated per mL. In some embodiments, the sample-probe incubations occur in a single well, e.g., a well of a microtiter plate, without inter-mixing between partitions. The microcapsules may also contain other components necessary for the incubation.

In some embodiments, the beads containing cells are partitioned into at least 500 partitions, at least 1000 partitions, at least 2000 partitions, at least 3000 partitions, at least 4000 partitions, at least 5000 partitions, at least 6000 partitions, at least 7000 partitions, at least 8000 partitions, at least 10,000 partitions, at least 15,000 partitions, at least 20,000 partitions, at least 30,000 partitions, at least 40,000 partitions, at least 50,000 partitions, at least 60,000 partitions, at least 70,000 partitions, at least 80,000 partitions, at least 90,000 partitions, at least 100,000 partitions, at least 200,000 partitions, at least 300,000 partitions, at least 400,000 partitions, at least 500,000 partitions, at least 600,000 partitions, at least 700,000 partitions, at least 800,000 partitions, at least 900,000 partitions, at least 1,000,000 partitions, at least 2,000,000 partitions, at least 3,000,000 partitions, at least 4,000,000 partitions, at least 5,000,000 partitions, at least 10,000,000 partitions, at least 20,000,000 partitions, at least 30,000,000 partitions, at least 40,000,000 partitions, at least 50,000,000 partitions, at least 60,000,000 partitions, at least 70,000,000 partitions, at least 80,000,000 partitions, at least 90,000,000 partitions, at least 100,000,000 partitions, at least 150,000,000 partitions, or at least 200,000,000 partitions.

Following tagging of target nucleic acids and also separate linking of cell-barcoding oligonucleotides to array oligonucleotides, the contents of the partitions can be combined and the remaining steps of sample preparation for sequencing can be performed “in bulk.” Both the tagged target nucleic acids and the cell-barcoding oligonucleotides linked to array oligonucleotides can be sequenced as desired.

Any method of nucleotide sequencing can be used as desired so long as at least some of the DNA segments sequence and the barcode sequence is determined. Methods for high throughput sequencing and genotyping are known in the art. For example, such sequencing technologies include, but are not limited to, pyrosequencing, sequencing-by-ligation, single molecule sequencing, sequence-by-synthesis (SBS), massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety.

Exemplary DNA sequencing techniques include fluorescence-based sequencing methodologies (See, e.g., Birren et al., Genome Analysis: Analyzing DNA, 1, Cold Spring Harbor, N.Y.; herein incorporated by reference in its entirety). In some embodiments, automated sequencing techniques understood in that art are utilized. In some embodiments, the present technology provides parallel sequencing of partitioned amplicons (PCT Publication No. WO 2006/0841,32, herein incorporated by reference in its entirety). In some embodiments, DNA sequencing is achieved by parallel oligonucleotide extension (See, e.g., U.S. Pat. Nos. 5,750,341; and 6,306,597, both of which are herein incorporated by reference in their entireties). Additional examples of sequencing techniques include the Church polony technology (Mitra et al., 2003, Analytical Biochemistry 320, 55-65; Shendure et al., 2005 Science 309, 1728-1732; and U.S. Pat. Nos. 6,432,360; 6,485,944; 6,511,803; herein incorporated by reference in their entireties), the 454 picotiter pyrosequencing technology (Margulies et al., 2005 Nature 437, 376-380; U.S. Publication No. 2005/0130173; herein incorporated by reference in their entireties), the Solexa single base addition technology (Bennett et al., 2005, Pharmacogenomics, 6, 373-382; U.S. Pat. Nos. 6,787,308; and 6,833,246; herein incorporated by reference in their entireties), the Lynx massively parallel signature sequencing technology (Brenner et al. (2000). Nat. Biotechnol. 18:630-634; U.S. Pat. Nos. 5,695,934; 5,714,330; herein incorporated by reference in their entireties), and the Adessi PCR colony technology (Adessi et al. (2000). Nucleic Acid Res. 28, E87; WO 2000/018957; herein incorporated by reference in its entirety).

Typically, high throughput sequencing methods share the common feature of massively parallel, high-throughput strategies, with the goal of lower costs in comparison to older sequencing methods (See, e.g., Voelkerding et al., Clinical Chem., 55: 641-658, 2009; MacLean et al., Nature Rev. Microbiol., 7:287-296; each herein incorporated by reference in their entirety). Such methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the Heli Scope platform commercialized by Helicos BioSciences, and platforms commercialized by VisiGen, Oxford Nanopore Technologies Ltd., Life Technologies/Ion Torrent, and Pacific Biosciences, respectively.

Once sequencing reads are generated from tagged nucleic acids and cell-barcoding oligonucleotides linked to array oligonucleotides, one can correlate the signal on the array, which indicates the phenotype of the cells, to the nucleotide sequence of the tagged nucleic acids from those same cells. Having recorded the location of the spots and phenotype signals from the spots, and knowing the sequences of the array oligonucleotides at the spots, one can correlate the resulting genotype information from the sequencing, i.e. the sequence reads from the cells. This can be achieved for example by identifying which array spot barcode is linked to which bead-specific barcode (i.e., from the cell-barcoding oligonucleotides linked to array oligonucleotides) and using this to deconvolute which cellular nucleic acid reads are from cells located in which spot on the array, thereby correlating phenotype signal to genotype.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of determining the phenotype and genotype of cells, the method comprising
providing a plurality of hollow hydrophilic beads, the beads containing a cell, wherein the beads are linked to a plurality of clonal cell-barcoding oligonucleotides having a 3′ capture sequence;
contacting the beads containing the cells to an array of spots of array oligonucleotides linked to a solid planar surface, wherein the spots have clonal copies of array oligonucleotides and different spots have unique array oligonucleotides such that the sequence of the array oligonucleotides identifies the spot and wherein the array oligonucleotides comprise a 3′ end sequence that is a reverse complement of the 3′ capture sequence of the cell-barcoding oligonucleotides, wherein the spots have a size that only accommodates a single cell such that single cells reside on single spots and wherein the sequence and location of the array oligonucleotides on the array is known;
assaying the cells on the spots to determine a phenotype and recording for the spots a signal indicative of the phenotype;
releasing the oligonucleotides from the array such that released oligonucleotides from spots diffuse into beads residing on the spots, wherein a portion of the cell-barcoding oligonucleotides anneal to the array oligonucleotides;
encapsulating the beads containing cells into aqueous droplets in a water-in-oil emulsion;
tagging in the droplets at least one cellular nucleic acid from the cells with the cell-barcoding oligonucleotides;
nucleotide sequencing tagged cellular nucleic acids from the cell and nucleotide sequencing cell-barcoding oligonucleotides linked to the array oligonucleotides, wherein the cell barcode on the cellular nucleic acid indicates the cell origin of the cellular nucleic acid and the array oligonucleotide indicates the location of the cell on the array; and
correlating the signal on the array to the nucleotide sequence of the tagged cellular nucleic acids.

2. The method of claim 1, after the providing and before the contacting, inducing the phenotype in the cells and sorting the beads for beads containing cells that generate the signal, thereby forming a population of beads enriched for beads containing cells, and wherein the contacting comprises contacting the population of beads enriched for beads containing cells to the array of spots of array oligonucleotides linked to the solid planar surface.

3. The method of claim 2, wherein the signal is a fluorescent signal and the sorting comprises sorting the cells with fluorescence-activated cell sorting (FACS)

4. The method of claim 1, wherein the tagging comprises annealing the 3′ capture sequence of the cell-barcoding oligonucleotides to the at least one cellular nucleic acid.

5. The method of claim 1, wherein the 3′ capture sequence is a poly T sequence comprising at least five contiguous deoxythymidines.

6. The method of claim 1, wherein the 3′ capture sequence is a gene-specific capture sequence.

7. The method of claim 1, wherein bridge oligonucleotides are present in the droplets and the tagging comprises annealing a first end of the bridge oligonucleotide to the cell-barcoding oligonucleotides and a second end of the bridge oligonucleotide to the cellular nucleic acid from the cell.

8. The method of claim 1, wherein the beads are linked to at least a first set and a second set of clonal cell-barcoding oligonucleotides having a 3′ capture sequence, wherein the first set and second set have different 3′ capture sequences and wherein the 3′ capture sequence of the first set anneals to the array oligonucleotides and (i) the 3′ capture sequence of the second set anneals to the at least one cellular nucleic acid or (ii) the 3′ capture sequence of the second set anneals to a first end of a bridge oligonucleotide and a second end of the bridge oligonucleotide anneals to the at least one cellular nucleic acid.

9. The method of claim 4, wherein the tagging comprises ligating the cell-barcoding oligonucleotides to the at least one cellular nucleic acid.

10. The method of claim 4, wherein the tagging comprises primer extension wherein the cell-barcoding oligonucleotides are extended by a polymerase using the at least one cellular nucleic acid as a template.

11. The method of claim 1, wherein the one or more cellular nucleic acids are RNA.

12. The method of claim 11, wherein the tagging comprises reverse transcription.

13. The method of claim 1, wherein the one or more cellular nucleic acids are DNA.

14. The method of claim 1, wherein the cells are B cells and the at least one cellular nucleic acid from the cells encodes at least a portion of an antibody variable region.

15. The method claim 1, wherein the cells are T-cells and the at least one cellular nucleic acid from the cells encodes at least a variable portion of a T-cell receptor.

16. The method of claim 1, wherein the assaying comprises in situ immunofluorescence, in situ immunohistochemistry, or in situ hybridization to produce the signal.

17. The method of claim 1, wherein the assaying comprises adding target cells to the array and assaying the ability of the cells in the hollow hydrophilic beads to alter a phenotype of the target cells.

18. The method of claim 1, wherein the beads containing the cells are encapsulated in a droplet before the assaying.
